(19) United States
(12) Patent Application Publication
(10) Pub. No.: US 2012/0046237 A1
(43) Pub. Date: Feb. 23, 2012

(54) COMPOSITIONS FOR PROTECTION AGAINST SUPERFICIAL VASODILATOR FLUSH SYNDROME, AND METHODS OF USE

(76) Inventor: Theoharis C. THEOHARIDES, Brookline, MA (US)

(21) Appl. No.: 13/112,675

(22) Filed: May 20, 2011

Related U.S. Application Data
(63) Continuation-in-part of application No. 11/651,161, filed on Jan. 10, 2007, now Pat. No. 7,759,307.
Application No. 11/651,161 is a continuation-in-part of application No. 10/811,828, filed on Mar. 30, 2004, now abandoned.
Application No. 10/811,828 is a continuation-in-part of application No. PCT/US02/00476, filed on Jan. 3, 2002.
Application No. PCT/US02/00476 is a continuation-in-part of application No. 09/771,669, filed on Jan. 30, 2001, now Pat. No. 6,984,667.
Application No. 09/771,669 is a continuation-in-part of application No. 09/056,707, filed on Apr. 8, 1998, now Pat. No. 6,689,748.
Application No. 09/056,707 is a continuation-in-part of application No. 11/999,991, filed on Dec. 10, 2007.
Application No. 11/999,991 is a continuation-in-part of application No. 11/651,161, filed on Jan. 10, 2007, now Pat. No. 7,759,307.
Application No. 11/651,161 is a continuation-in-part of application No. 10/811,828, filed on Mar. 30, 2004, now abandoned.

(51) Int. Cl. A61K 31/70; A61K 31/4545; A61K 31/445; A61P 37/08; A61K 31/352; C07D 311/02; A61P 15/00; A61P 9/00; A61K 31/517; A61K 31/403

(52) U.S. Cl. 514/25; 514/266.21; 514/318; 514/325; 514/411; 514/456; 549/398

(57) ABSTRACT

Compositions for protection against SVFS induced by niacin, a carcinoid, mesenteric traction, serotonin, post-menopause, alcohol, monosodium glutamate, mastocytosis, atopic dermatitis, food-allergy or food intolerance, and mast cell activation syndrome, or against individual symptoms of SVFS, superficial vasodilation, feeling of warmth, itching (pruritus) and hives, comprising a flavonoid compound of the structure 2-phenyl-4H-1-benzopyran or 2-phenyl-4-keto-1-benzopyran or glycosides thereof, or chalconoid compounds, with appropriate substitutions of their hydroxyl groups to render them water soluble or in combination with a pshospholipid or cyclodextrin to render them to have higher oral absorption, administered alone or together with an anti-superficial vasodilation dose of one or more of, olive kernel oil, a serotonin inhibitor, a prostaglandin inhibitor, willow bark extract. A composition for treating cardiovascular disease with niacin, but without eliciting the SVFS effects of niacin, has also been invented.

### CROSS-REFERENCES TO RELATED APPLICATIONS

This application is a continuation-in-part application of co-pending U.S. Ser. No 11/999,991, filed Dec. 10, 2007, which was a continuation-in-part application of co-pending U.S. Ser. No. 11/651,161, filed Jan. 10, 2007, which was a CIP of co-pending U.S. Ser. No. 10/811,828, filed Mar. 30, 2004. This application is a continuation-in-part application of U.S. patent application Ser. No. 11/651,161, filed Jan. 10, 2007, which is a continuation-in-part application of U.S. patent application Ser. No. 10/811,828, filed Mar. 30, 2004, which is a continuation-in-part application of PCT/US02/00476, filed Jan. 3, 2002, which is a continuation-in-part application of U.S. patent application Ser. No. 09/771,669, filed Jan. 30, 2001, now U.S. Pat. No. 6,984,667, which is a continuation-in-part application of U.S. patent application Ser. No. 09/056,707, filed Apr. 8, 1998, now U.S. Pat. No. 6,689,748.

### BACKGROUND OF THE INVENTION

The invention is generally related to the treatment of superficial vasodilator flush syndrome (“SVFS”). More specifically, the invention relates to compositions containing inhibitors of superficial vasodilators such as niacin, histamine, prostaglandins and serotonin, for example, a flavonoid compound, alone or together with other inhibitors of superficial vasodilators such as a PGD₂ or serotonin inhibitor, that are designed to be used as dietary supplements to conventional approved medications for protection against SVFS. An inhibitor is defined as any compound that can block the action of PGD₂ or serotonin either through inhibition of their release, through their neutralization or through antagonism of their respective receptors.

In spite of risk factors, better recognition and availability of more efficacious drugs for lowering serum cholesterol and triglycerides, mortality from cardiovascular disease continues to occur in ⅔ of patients treated with statins, and to increase worldwide by about 25% (Libby, P, Amer. Coll. Cardiol. 46:1225 (2005). Niacin (nicotinic acid) at 1-2 g/day, decreases low-density lipoprotein (“LDL”) and triglycerides, while increasing high-density lipoprotein (“HDL”) levels (Carlson, L A, J. Intern. Med. 258:94 (2005). Moreover, niacin and a statin together have superior lipoprotein lowering profile (Brown B G, et at, N. Eng. J. Med. 345:1583 (2001), as also shown for slow release niacin combined with lovastatin (Gupta E K et a/., Heart. Dis. 4:124 (2002). However, a limiting adverse effect in patients receiving immediate or sustained release niacin is the rapid development of significant cutaneous warmth and itching, especially on the face, referred to as “flush,” that severely limits compliance. (Gupta et al., supra).

Niacin-induced flush is thought to involve the release of prostaglandin D2 (PGD₂) from the skin (Morrow J D et al., J. Invest. Dermatol. 98:812 (1992); Morrow J D et al., Prostaglandins 38:263 (1989), especially from macrophages (Meyers C D et al., Atherosclerosis (2006); Urade Y. et al., J. Immunol. 50:191 (1989). However, co-administration of acetylsalicylic acid (ASA) to reduce PGD₂ levels has not been particularly effective (<30%) in blocking niacin flush (Dunn R T et al., J. Therap. 2:478 (1995); Cefali E A et al., Int. J. Clin. Pharmacol. Ther. 45:78 (2007). Consequently, molecules other than PGD₂ may be involved, such as histamine, vasoactive intestinal peptide (VIP) and vascular endothelial growth factor (VEGF) (Grutzkau A. et al., Mol. Cell Biol. 9:875 (1998); Boesiger J. et al., J. Exp. Med. 188:1135 (1998), as well as serotonin released from platelets, enterochromaffin cells (Boushey R P et al., Curr. Treat. Opt. 49:355 (2002), and mast cells (Kushnir-Sukhov N M et al., J. Allerg. Clin. Immunol. 119:498 (2006). Serotonin is a prime candidate because it is known to be involved in the flush associated with carcinoid syndrome (Boushey 2002, supra).

SVFS is not limited to niacin-induced flush and includes more symptoms than just superficial vasodilation, such as feeling of warmth, itching (pruritus) and hives. This syndrome is present in a number of other human conditions that includes carcinoid-induced, mesenteric traction-induced, serotonin-induced, postmenopause-induced, alcohol-induced, monosodium glutamate-induced, mastocytosis-induced, atopic dermatitis-induced, food-allergy or food intolerance-induced, and mast cell activation syndrome-induced SVFS.

An important need, therefore, exists for compositions for administration to humans suffering from SVFS produced by a variety of etiologies. This need is particularly urgent in patients suffering from niacin-induced SVFS, particularly those suffering with coronary artery disease who must reduce serum triglycerides and LDL cholesterol, and who cannot tolerate niacin alone or together with statins. Such formulations have now been discovered, and are described below.

### SUMMARY OF THE INVENTION

The invention comprises anti-SVFS compositions for human use that consist of a flavonoid compound having the basic structure of 2-phenyl-4H-1-benzopyran or its 4-keto counterpart or their glycosides, used either alone or together with one or more of a group comprising superficial vasodilation inhibitors, olive kernel oil (“OKO”), a prostaglandin inhibitor, a serotonin inhibitor, willow bark extract, S-adenosylmethionine (“SAMe”), histamine-1 receptor antagonists, histamine-3 receptor agonists, antagonists of the actions of corticotropin releasing hormone (“CRH”), caffeine, folic acid, polyunsaturated fatty acids, and polyamines, together with appropriate excipients and carriers, said compositions having improved anti-SVFS effects synergistic with each other and synergistic with available conventional clinical treatment modalities.

In preferred embodiments the flavonoid compound is luteolin, quercetin, genistein, myricetin and/or their respective glycosides.

In some embodiments, the flavonoid has its hydroxyl groups substituted with moieties that increase its water solubility.

In some embodiments, the flavonoid is formulated with a phospholipid or cyclodextran to increase oral absorption.

Where the serotonin inhibitors are comprised of prochlorperazine, cyproheptadine, azatadine and ketanserin.

In yet another embodiment, inventive compositions that protect humans against a variety of SVFS entities include a flavonoid compound alone or in combination with one or more of OKO, willow bark extract, a serotonin inhibitor a PGD2 inhibitor, and a CRH inhibitor.

The novel OKO may be used to increase the absorption of difficult to absorb flavonoids or drugs across the oral, gastric, intestinal, or nasal mucosa or pulmonary alveoli.

In a preferred embodiment of the invention, a composition for treating cardiovascular disease has been devised that includes niacin but does not elicit the SVFS that normally accompanies administration of this drug.

The invention further comprises a pharmaceutical composition for protecting humans against superficial vasodilator flush syndrome (“SVFS”) comprising a flavonoid compound of Formula I:

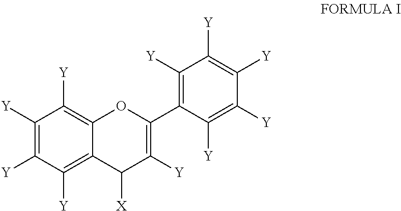

wherein X is carbonyl, O-Z, Y, or H;
each Y is independently H, O-Z, or a halogen;
Z is H, alkali metal, or alkali earth metal;
or a glycoside thereof.

In a preferred embodiment, the flavonoid compound is selected from the group consisting of luteolin, quercetin, myricetin, genistein, curcumin, epigallocatechin or a glycoside derivative of the flavonoids.

In a preferred embodiment, the composition comprises a compound that lowers total serum cholesterol or LDL cholesterol.

In a preferred embodiment, the cholesterol-lowering compound is a statin. In some embodiments, the statin is selected from the group consisting of simvastatin, lovastatin, atorvastatin, rosuvastatin, fluvastatin, and provastatin.

In a preferred embodiment, the composition comprises of a compound that increases serum HDL cholesterol.

In a preferred embodiment, the composition contains a prostaglandin inhibitor.

In a preferred embodiment, the prostaglandin inhibitor is selected from the group consisting of non-steroidal anti-inflammatory drugs, corticosteroids, cyclooxygenase-2 (COX-2) inhibitors, and PGD₂ antagonists.

In a preferred embodiment, the prostaglandin inhibitor is a PGD₂ inhibitor. In some embodiments, the PGD2 antagonist is laropiprant.

In a preferred embodiment, the flavonoid composition is supplemented with one or more additional anti-SVFS compounds.

In a preferred embodiment, the additional anti-SVFS compound is a serotonin inhibitor.

In a preferred embodiment, the inhibitor is a serotonin receptor antagonist.

In a preferred embodiment, the serotonin antagonist is prochlorperazine or ketanserin.

In a preferred embodiment, the serotonin inhibitor is a mixed serotonin receptor antagonist and histamine-1 receptor antagonist selected from the group consisting of cyproheptadine or azatadine.

The invention further comprises a method for protecting an individual from the SVFS effects induced by niacin intake comprising administration to said individual effective doses for effective periods of time of any one or more of the compositions of the present invention.

The invention further comprises a method for protecting an individual from SVFS-associated symptoms of superficial vasodilation, feeling of warmth, itching (pruritus) and hives.

The invention further comprises a method for protecting an individual from the SVFS effects associated with carcinoid-associated flush, mesenteric traction-induced flush, serotonin-induced flush, post-menopausal-induced flush, alcohol-induced flush and monosodium glutamate-induced flush, mastocytosis-induced, atopic dermatitis-induced, food-allergy or food intolerance-induced, and mast cell activation syndrome-induced SVFS, comprising administration to said individual effective doses for effective periods of time of any one or more of the compositions of the present invention.

The invention further comprises a method for treating a cardiovascular condition in a patient with niacin, but without eliciting the SVFS effect of said niacin, comprising the administration to said patient of clinically effective amounts of the compositions of the present invention.

The invention further comprises a pharmaceutical composition for protecting humans against superficial vasodilator flush syndrome (“SVFS”) comprising a chalconoid compound of Formula II:

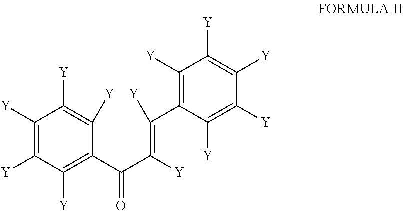

wherein each Y is independently H, O-Z, or a halogen;
Z is H, alkali metal, or alkali earth metal;
or a glycoside thereof.

In a preferred embodiment, the flavonoid compound is selected from the group consisting of luteolin, quercetin, myricetin, genistein, curcumin, epigallocatechin or a glycoside derivative of the flavonoids.

In a preferred embodiment, the pharmaceutical composition contains a composition that lowers total serum cholesterol or LDL cholesterol.

In a preferred embodiment, the composition comprises of a compound that increases serum HDL cholesterol.

In a preferred embodiment, the cholesterol-lowering composition is a statin.

In a preferred embodiment, the statin is selected from the group consisting of simvastatin, lovastatin, atorvastatin, rosuvastatin, fluvastatin, and provastatin.

In a preferred embodiment, the pharmaceutical composition contains a prostaglandin inhibitor.

In a preferred embodiment, the prostaglandin inhibitor is selected from the group consisting of non-steroidal anti-inflammatory drugs, corticosteroids, and cyclooxygenase-2 (COX-2) inhibitors.

In a preferred embodiment, the chalconoid composition is supplemented with one or more additional anti-SVFS compounds.

In a preferred embodiment, the supplemental anti-SVFS compound is a serotonin inhibitor.

In a preferred embodiment, the inhibitor is a serotonin receptor antagonist.

In a preferred embodiment, the serotonin antagonist is prochlorperazine or ketanserin.

In a preferred embodiment, the serotonin inhibitor is a mixed histamine-1 and serotonin receptor antagonist selected from the group consisting of cyproheptadine or azatadine.

The invention further comprises a method for protecting an individual from the SVFS effects induced by niacin intake comprising administration to said individual effective doses for effective periods of time of any one or more of the pharmaceutical compositions comprising a chalconoid.

The invention further comprises a method for protecting an individual from the SVFS effects associated with carcinoid-associated flush, mesenteric fraction-induced flush, serotonin-induced flush, post-menopausal-induced flush, alcohol-induced flush, monosodium glutamate-induced flush, mastocytosis-induced, atopic dermatitis-induced, food-allergy or food intolerance-induced, and mast cell activation syndrome-induced flush comprising administration to said individual effective doses for effective periods of time of any one or more of the pharmaceutical compositions comprising a chalconoid.

A method for treating a cardiovascular condition in a patient with niacin, but without eliciting the SVFS effect of said niacin, comprising the administration to said patient of clinically effective amounts of a pharmaceutical composition comprising a chalconoid.

### BRIEF DESCRIPTION OF THE DRAWINGS

FIG. 1. (A) A time-course of ear temperature increase (n=5) in response to a single intraperitoneal niacin (7.5 mg/rat) injection. All time points were significant (p=0.0002). (B) Dose-response of the effect of a single ip niacin injection on ear temperature increases recorded 45 min later (n=5). Niacin rat doses were based on 80 kg human (H) doses as follows: 5.0 mg/rat=1,167 mg/H; 7.5 mg/rat=1,750 mg/H; 10 mg/rat=2,334 mg/H (p=0.0001).

FIG. 2. Comparison of the inhibitory effect of fisetin, kaempferol, luteolin, myricetin, quercetin (4.3 mg/rat =1000 mg/80 kg), and ASA (1.22 mg/rat), administered ip 10 min prior to niacin on the ear temperature increase recorded 45 min after a single intraperitoneal injection of niacin (7.5 mg/rat) in olive oil (n=6, *p=0.0204, **p=0.0041, ***p=0.0002, ****p=0.0193). The percent inhibition was calculated after the corresponding baseline temperature was subtracted.

FIG. 3. Time course of the inhibitory effect of luteolin 4.3 mg/rat pre-treatment (0=luteolin added together with niacin) on ear temperature increase (n=3) in response to a single ip injection of niacin (7.5 mg/rat) measured 45 min later. All time points were statistically significant as compared to a control rat injected with 0.5 ml olive oil and 7.5 mg niacin. Brackets indicate groups compared (*p<0.001).

FIG. 4. Effect of acetylsalicylic acid (“ASA”) (1.22 mg/rat) and luteolin (4.3 mg/rat) administered 2 hr prior to a single ip injection of niacin (7.5 mg/rat) on plasma PGD₂ levels measured 45 min later (n=3). Bracket indicate groups compared (*p=0.014; **p=0.0419).

FIG. 5. Effect of ASA (1.22 mg/rat) and luteolin (4.3 mg/rat) administered 2 hr prior to a single ip injection of niacin (7.5 mg/rat) on plasma serotonin levels measured 45 min later (n=3). Brackets indicate groups compared (*p=0.0263).

FIG. 6. Effect of aspirin (ASA; 1.22 mg per rat) and luteolin (4.3 mg per rat) administered 2 h prior to a single i.p. injection of niacin (7.5 mg per rat) on plasma 5-HT levels measured 45 min later (n=3). Brackets indicate groups compared (*P=0.0263).

FIG. 7. At the same concentration, water soluble quercetin shows better inhibitory effects compared with anhydrous quercetin dissolved in DMSO (n=3; * indicates such difference is statistically significant at p<0.05). Beta-Hex release parallels histamine release and serves a measurement of mast cell degranulation. The higher the value, the more pro-inflammatory mediators such as histamine is released from mast cells. Substance P (SP) is a neuropeptide that is commonly used to stimulate mast cells.

FIG. 8. At the same concentration, water soluble quercetin shows better inhibition of tumor necrosis factor (TNF), an important cytokine involved in inflammation, compared with anhydrous quercetin dissolved in DMSO (n=3; * indicates such difference is statistically significant at p<0.05).

### DETAILED DESCRIPTION OF THE PREFERRED EMBODIMENTS

It has been discovered that a flavonoid compound, a chalconoid, or their glycoside counterparts, either alone or in combination with one or more of a group of vasodilation inhibitors consisting of OKO, a serotonin inhibitor, a prostaglandin inhibitor, a willow bark extract, S-adenosylmethionine (“SAMe”), a CRH inhibitor, a histamine-1 receptor antagonist, a histamine-3 receptor agonist, a polyamine, rutin and caffeine, have synergistic anti-SVFS effects, where SVFS is caused by to ingestion of niacin or other SVFS inducers listed supra. OKO may be used to improve the transmembrane transport of difficultly-absorbable drugs in the intestine, skin, nasal, oral and pulmonary alveoli.

The preferred flavonoid compounds are luteolin and quercetin. In addition, other flavonoid compounds suitable in carrying out the invention include the quercetin glycoside rutin, myricetin, kaempferol glycoside astragaline, genistein, kaempferol, curcumin, epigallocatechin and the isoflavone phenoxodiol.

The OKO component of the inventive compositions is, preferably, an unrefined (first pressing, filtered, oleic acid-related acidity<3%, water content<1%) oil produced, for one source, on the island of Crete in Greece. This olive kernel oil product is especially prepared by applicant's process consisting essentially of: (1) harvesting first collection ripe olives, preferably in December; (2) compressing the oil from the flesh of the ripe olives; (3) washing the kernels remaining after step (2) with water to remove debris; (4) drying the washed kernels with a stream of hot air; (5) crushing the dried kernels to produce an oil; (6) removing participate matter from the organic extract by centrifugation or microfiltering through 1-2 micron pore size filters; (7) evaporating any water by raising the temperature to 86-100 ° C., which reduces the water content to <1%, the acidity (as oleic acid) to <3%; and, the organic solvent to <1%; and (8) storing the final kernel extract product in the absence of air.

The inventive OKO surprisingly has the unique property of increasing absorption of the flavonoids of the anti-SVFS compositions through the intestinal mucosa or skin, and also adds its own content of important anti-oxidants, such as omega fatty acids (e.g., eicosapentanoic acid) and alpha tocopherol. The polyphenols found in such OKO also have anti-inflammatory effects in, for example, arthritis [Martinez-Dominguez et al., Inflamm. Res. 50:102 (2001)]. E.B.E.K., Inc., Commercial, Industrial Enterprises of Crete, 118 Ethnikis Antistasecos, Heraklion, Crete, 71306, Greece, or MINERVA Edible Oils, 165 Tatoiou St., Athens, 14452, Greece, will prepare the OKO according to applicant's above-described procedure for commercial users. Parallel experiments with codfish oil, corn oil and olive oil (from the flesh of the olive) were contemplated, but flavonoid sulfate solubility in these oils was insufficient to meet the requirements of the experiment.

In addition to its usefulness in increasing the absorption of the inventive compositions across the intestinal wall and the skin, the inventive OKO product is useful in aiding the dissolution of other drugs prior to administration to a patient, and is useful in promoting the absorption of other difficult to absorb drugs across oral mucosa, gastric mucosa, intestinal mucosa, nasal mucosa, skin and lung alveoli of patients.

In experiments with rat models of the SVFS, to be described in detail infra, applicant has surprisingly also discovered that serotonin mostly mediates the flush syndrome induced by niacin administration. This discovery has opened up a new therapeutic approach for niacin flush. Applicant has discovered that serotonin inhibitors such as prochlorperazine, cyproheptadine, azatadine and ketanserin, when used alone or in combination with the basic composition of the invention, inhibit the niacin flush syndrome

Another optional supplement to the basic compositions of the invention is a histamine-1 receptor antagonist, such as hydroxyzine, mezelastine. azelastine. azatadine, rupatadine. Other histamine-1 receptor antagonists are described in Table 25-1 in Goodman and Gilman's The Pharmaceutical Basis of Therapeutics, 9^{th} ed., New York, 1996. Histamine-3 receptor agonists are described in the Theoharides patents listed above.

The preferred concentration range of the flavonoid components of the oral formulations are 50-3,000 mg per tablet or capsule. Generally, where present, the amounts of OKO are somewhat less to equal to those of the other active ingredients, preferably 50-1500 mg. The number of capsules or tablets to be taken per day is determined by the nature and severity of the medical condition, and is readily determinable by the patient's health provider, one preferred dosing being two capsules per 20 kg body weight. Other representative formulations are described in the examples infra.

The anti-SVFS compositions of the invention may be used together with serum cholesterol- and LDL-lowering statins, such as simvastatin, lavastatin, atorvastatin, rosuvastatin, fluvastatin, pravastatin, or with compounds increasing HDL.

The anti-SVFS composition of the invention may be used together with a prostaglandin inhibitor, such as non-steroidal anti-inflammatory drugs, COX-2 inhibitors, corticosteroids.

The compositions of the present invention comprising Formula I are flavonoids with, or without, halogen, alkali, or alkali earth metal substitutions to the core flavonoid structure. The compositions of the present invention comprising Formula II are chalconoids with, or without, halogen, alkali, or alkali earth metal substitutions to the core chalconoid structure.

The compositions of the invention may be formulated in phospholipid or cyclodextrin to increase oral absorption, or in any standard means of introducing Pharmaceuticals into a patient, e.g., by means of tablets or capsules. Standard excipients and carriers for the active ingredients of the inventive compositions are described in Remington's Pharmaceutical Sciences, Mack Publishing Co., Easton, Pa.

### EXAMPLES

### Example 1

### Treating Niacin-Flush in Humans

Four normal male subjects (29±3 years) were entered in the following protocol: On days 1 and 2, they were administered 1 gm immediate release niacin, at 2 pm. On days 3 and 4 they were administered 2 capsules of a composition containing 150 mg quercetin and 450 mg of OKE per capsule. On days 4 and 6, they were administered two capsules at 8 am and 1 g niacin at 2 pm. Skin temperature was measured with an infrared digital pyrometer at 4 facial sites (forehead, both checks and chin) at 15, 30, 45, 60, 75 and 90 min post niacin administration, along with daily room temperature subjects also completed a symptoms questionnaire (erythema, edema, pruritus and burning sensation) on a scale of 0=no symptoms and 5=maximum symptoms. There was no significant increase in temperature rise with niacin administration, but symptoms (especially erythema and burning) ranged 4-5 and lasted 3-4 hrs. After administration of the inventive composition, the scores were reduced to 2-3 and lasted only about 75 min. (>50% inhibition). These results demonstrate that the inventive compositions containing a flavonoid reduce niacin flush.

| | Niacinalone | | Niacin + Inventive Composition | |
|---|---|---|---|---|
| Erythema | 4.75 ± 0.5 | 4.5 ± 0.58 | 3.25 ± 0.5 | 2.5 ± 0.58 |
| Edema | 0.5 ± 0.58 | 0.5 ± 0.58 | 0.25 ± 0.5 | 0.25 ± 0.5 |
| Urticaria | 2.25 ± 0.5 | 2.0 ± 0.82 | 1.75 ± 0.5 | 1.25 ± 0.5 |
| Burning | 4.75 ± 0.5 | 4.0 ± 0.82 | 3.0 ± 0.82 | 2.5 ± 0.58 |
| Duration | 3.63 ± 1.11 | 2.75 ± 0.87 | 1.68 ± 0.40 | 1.68 ± 0.70 |
| (hr) | | | | |

### Example 2

### Protection Against Niacin Flush in an Animal Model

Materials and Methods—Male Sprague-Dawley rats (300-350 g) were housed three per cage and were provided with food and water ad libitum. The room temperature was kept constant at 21±1° C., with a 14:10 hour light/dark schedule and lights out at 19:00 hour. ASA, fisetin, kaempferol, luteolin, myricetin, niacin, and quercetin were purchased from Sigma (St. Louis, Mo.). All drugs were first dissolved in OKE and then 0.9% NaCl fresh each day of the experiment.

Assessment of niacin-induced skin temperature changes—Temperature measurements were recorded with a hand-held infrared pyrometer connected to a millivoitmeter (Model OS613A, Omega Co., Stamford, Conn.). The probe was held at a distance of 1-2 mm from the animal's skin and temperature readings were taken from an ear area approximately 3 mm in diameter. Animals were habituated to handling and to the infrared probe for 3 days before use. On the day of the experiment, the animals were brought into the lab (9-10 AM). Three temperature readings from the top half of each ear were recorded for each time point without anesthesia immediately before animals were injected intraperitoneally (ip) with either niacin or the test flavonoid. The ear temperature was then measured every 10 min for a period up to 60 min. The animals were returned to their cages between measurements. Animals were “rested” for one week and were used again; the effect of niacin was not changed in rats that were used more than once.

Pre-treatment with various flavonoids—Rats were randomly administered either (A) vehicle (olive kernel extract) followed by niacin or (B) a flavonoid (4.3 mg/rat, equivalent to 1,000 mg/80 kg human) followed by niacin. This dose of flavonoids, the structures of which differs only by 1 hydroxyl group at certain positions, was chosen because it was previously shown to be attainable in vivo (Kimata et a/., 2000a) by oral administration.

Blood mediator measurements—In certain cases, blood was collected immediately after the end of the experimental period by sacrificing the animal wish asphyxiation over CO₂ vapor decapitation and collection from neck vessels. Blood was centrifuged at 350×g in a refrigerated centrifuge, the plasma collected and frozen at −20° C. until assay. Plasma levels of PGD₂ (Cayman) and serotonin (Biosource, Belgium) were assayed by ELISA kit (Biosource, Belgium). The lowest levels of sensitivity for each were 200 pg/ml (intra and inter-assay variation 10-20%) and 0.5 ng/ml (intra-assay variation 26 and inter-assay variation 15), respectively.

Statistical analysis—The six ear temperature measurements (three from each ear) were averaged for each point. Any temperature change was calculated by subtracting from the mean value for each experimental point the baseline temperature obtained immediately before the vehicle/drug was injected or the baseline measured immediately before niacin administration, whichever was appropriate. All data are presented as mean±SD of the actual temperatures or percent change from that recorded after niacin administration. Paired comparisons between niacin and control or niacin and drug pretreatment followed by niacin were analyzed with either the paired t-test or the non-parametric Mann-Whitney U test. Multi-variant ANOVA analysis was performed on all other comparisons. Significance is denoted by p<0.05. Niacin was administered to unanesthetized rats, using 3 animals per dose.

A: Effect of niacin on skin temperature

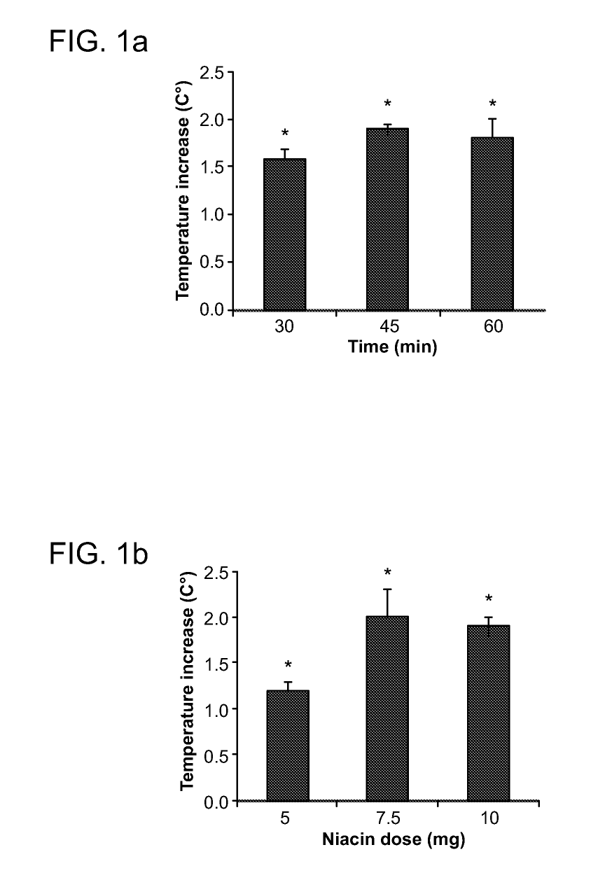

The basal mean ear temperature was 26.5-28.5 C (n=27). Niacin (7.5 mg/rat, equivalent to 1,750 mg/80 kg human) administered ip in conscious rats induced a time-dependent temperature increase with a maximum 1.9±0.2° C. (n=5, p=0.0002) at 45 min (FIG. 1A). A dose-response of niacin (5-10 mg/rat, n=5) showed maximal temperature increase of 2.0±0.1° C. (p=0.001) at 45 min with 7.5 mg/rat (FIG. 1B).

B: Treatment with azatadine (histamine-1 receptor antagonist) and serotonin receptor antagonist.

Rats were treated with 1 μg of azatadine i.p. at time zero. Niacin, 5 mg, was given i.p. 45 mins. mins. post-azatadine, and ear temperatures were measured.
At 10 mins., azatadine had reduced the niacin +2 degrees C. effect by 75%.

C: Treatment with cyproheptadine (strong histamine H1 and serotonin receptor antagonist)

Rats were treated with 8.55 μg of the antagonist i.p. at time zero, and niacin, 5 mg, was given at 120-480 mins. thereafter. Ear temperatures were measured at 45 mins. after niacin. There was no effect of niacin in animals pre-treated with cyproheptadine (100% inhibition).

D: Treatment with ketotifen (histamine-1-receptor antagonist)

Rats were pretreated with 17.1 μg of ketotifen, and niacin, 5 mg, was administered i.p. 30 mins. thereafter. Ear temperatures were measured 45 mins. after niacin.
The drug had no significant effect on the niacin effect.

E: Treatment with Quercetin.

Quercetin, 4.7 mg, was given to rats i.p. at time zero, and 5 ng niacin administered i.p. 120, 240 and 360 mins. thereafter.
Quercetin inhibited the niacin effect by 100%.

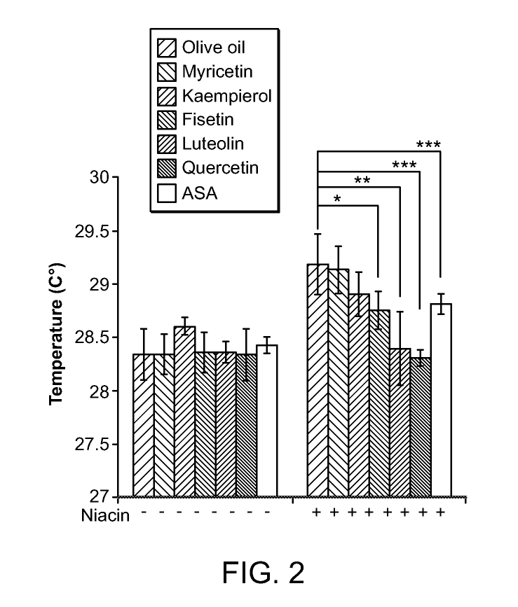

F: Effect of ASA and flavonoids on niacin-induced skin temperature increase We investigated whether pretreatment for 2 hr with ASA (1.22 mg/rat equivalent to 325 mg/80 kg human) or various flavonoids (4.3 mg/kg, equivalent to 1,000 mg/80 kg human) could inhibit niacin's effect (7.5 mg/rat) in this animal model. ASA inhibited this effect by 30% (n=6, p=0.0193, FIG. 2). Myricetin and kaempferol had no effect; fisetin inhibited the effect of niacin by 50% (n=6, p=0.0204, FIG. 2). Quercetin and luteolin were the most effective in reducing ear temperatures by 96% and 88%, respectively (n=6, p=0.0002 and p=0.0041, FIG. 2); there was no statistical difference between the effects of quercetin and luteolin.

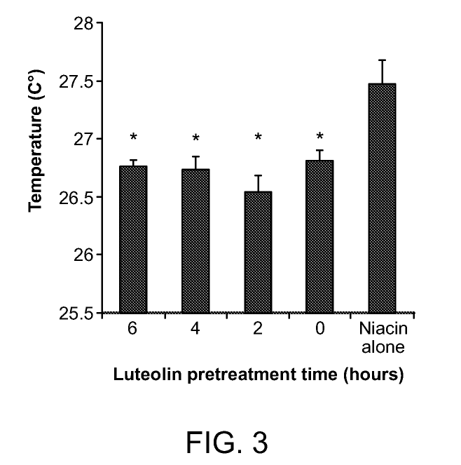

G: Effect of pretreatment duration with luteolin We then investigated whether the length of pretreatment with luteolin affected its ability to inhibit the niacin flush. Luteolin significantly decreased the niacin induced temperature increase even when added together with niacin (time=0) and remained significant at all time points from 0 to 6 hours. There was no significant difference between the 2, 4, and 6 hour pretreatment time points amongst the luteolin pretreated samples (FIG. 3).

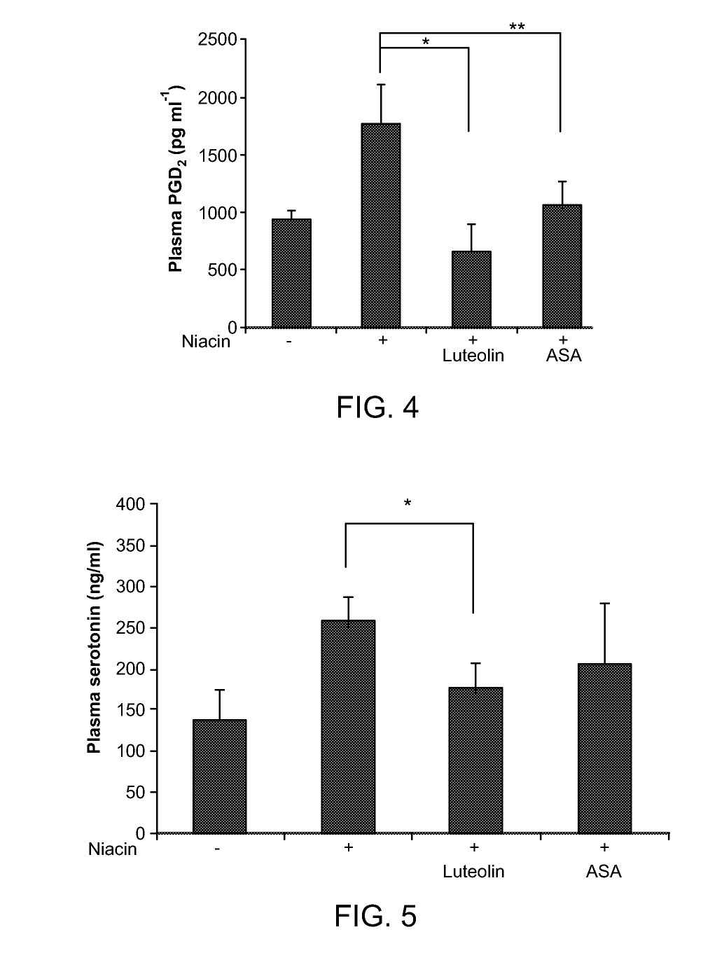

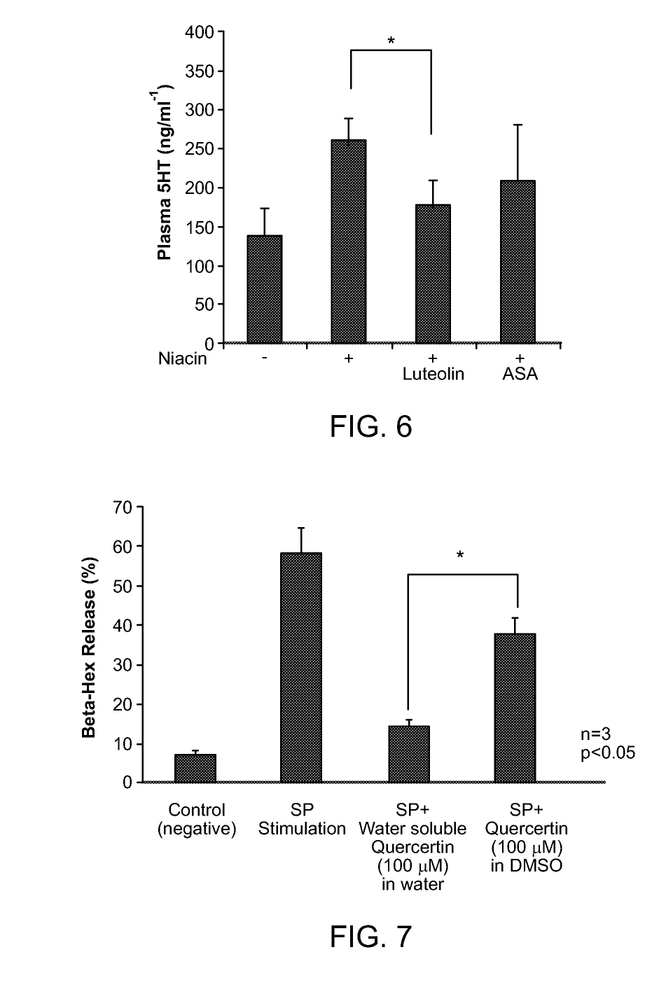

H: Effect of niacin on plasma PGD₂ and serotonin levels We then investigated the effect of niacin, as well as the effect of the luteolin, on niacin-induced plasma PGD2 and serotonin levels. Niacin (7.5 mg/rat) increased plasma PGD₂ by 88% from 933194 pg/ml to 1750±352 pg/ml at 45 min (n=3, p=0.0178, FIG. 4) and plasma serotonin by 90% from 137±37 ng/ml (n=4) to 260±28 ng/ml (n=4, p=0.0101, FIG. 5).

I: Effect of ASA and luteolin on niacin-induced plasma PGD₂ and serotonin levels Pretreatment for 2 hr with ASA (1.22 mg/rat) reduced plasma PGD₂ by 86% (n=3, p=0.018, FIG. 4), but had no statistically significant inhibitory effect on plasma serotonin levels (FIG. 5). In contrast, luteolin (4.3 mg/rat) significantly reduced plasma PGD₂ levels by 100% (n=3, p=0.014, FIG. 4), and serotonin levels by 32% (n=3, p=0.0263, FIG. 5). If the baseline serotonin of 137 ng/ml were to be subtracted from the niacin-induced level of 260 ng/ml and the level of 177 ng/ml in the presence of both luteolin and niacin, and then calculate the inhibition, it now becomes 97%.

### Example 3

A Representative Example of a Composition
for Protecting Against SVFS

| Ingredients. | per capsule: | |
|---|---|---|
| *Luteolin | 250 | mg |
| Optionally: | | |
| *Olive kernel oil | 450 | mg |
| *Willow bark extract | 100 | mg |
| *Cyproheptadine or azatadine | 4 | mg |

***

### Example 4

A Representative Composition for Treating Cardiovascular
Disease That Contains Niacin But Does Not Exhibit SVFS

Amount per 2 #120 Softqel Capsules*

| | | |
|---|---|---|
| Luteolin | 300 | mg |
| Niacin | 300 | mg |
| S-adenosylmethionine | 200 | mg |
| Folic acid | 140 | μg |
| Eicosapentenoic Acid or | 100-200 | mg |
| Docosahexenoic acid | | |
| Optionally: | | |
| OKE | 50-1500 | mg |
| Willow bark extract | 200 | mg |

*The number of capsules to be taken per day will depend on the status of the cardiovascular condition in the patient.

### Example 5

### Effect of Olive Kernel Extract on Absorption of a Flavonoid in Vivo

Quercetin (98% pure from Saphora Japonica) was tritiated by New England Nuclear Corp. to a specific activity of 4.9 mCi/ml.

Unlabeled quercetin was dissolved in OKO (55% w/v to about 45% w/v of OKO (2.5% acidity as oleic acid, 1.0% water). To this solution was added 20.2 microCuries of the labeled quercetin. AAA gelatin capsules were filled with the resulting solution using an aluminum template molding device.

The laboratory animals (250 g male Sprague-Dawley rats) were kept overnight without food, but with free access to water. Two capsules/100 g weight containing the above-described quercetin-OKO solution were given to each rat by gavage. Control animals were given the equivalent amount of anhydrous quercetin, but without OKO. The animals were then given free access to food. Blood radioactivity was measured 8 hours thereafter using a beta scintillation counter.

The results showed that, in control animals, about 4.9%+/−1.4% (n=3) of the dose of labeled quercetin reached the circulation. In sharp contrast, in animals given the labeled quercetin in OKO, about 17.9%+/−2.7% (n=4) of the dose was absorbed into the general circulation.

These results demonstrate that OKO increased by almost 4-times the absorption of quercetin, as compared to quercetin powder, from the intestine into the general circulation.

### Example 6

### Effect of Aspirin or Flavonoids on Serum PGD2 Levels Induced by Niacin

Effect of ASP (1.22 mg/rat) and luteolin (4.3 mg per rat) administered i.p. 2 hrs previously to 7.5 mg/rat of niacin to a rat, and plasma PGD2 was measured at the 45″ time point. Luteolin reduced the SVFS effect of niacin by about 50% at 45″.

ASP had a substantially lesser effect on SVFS than did luteolin (FIG. 7).

### Example 7

### Effect of Water Solubility on the Effectiveness of Quercetin at Suppressing Histamine Release

As can be seen in FIG. 7, increasing the water solubility of quercetin, increases the effectiveness of the flavanoid in suppressing beta-hex release. Beta-hex release parallels histamine release and serves as a measurement of mast cell degranulation. The higher the value, the more pro-inflammatory mediators, such as histamine, are released from mast cells. In the experiment in FIG. 7, an equivalent amount (300μ) of anhydrous quercetin in Dimethyl sulfoxide and water soluble quercetin in water were administered. At the same concentration, water soluble quercetin shows better inhibitory effects compared with quercetin dissolved in DMSO. Substance P (SP) is a neuropeptide that is commonly used to stimulate mast cells serves as a positive control.

### Example 8

### Effect of Water Solubility on the Effectiveness of Quercetin at Suppressing TNF-alpha Secretion

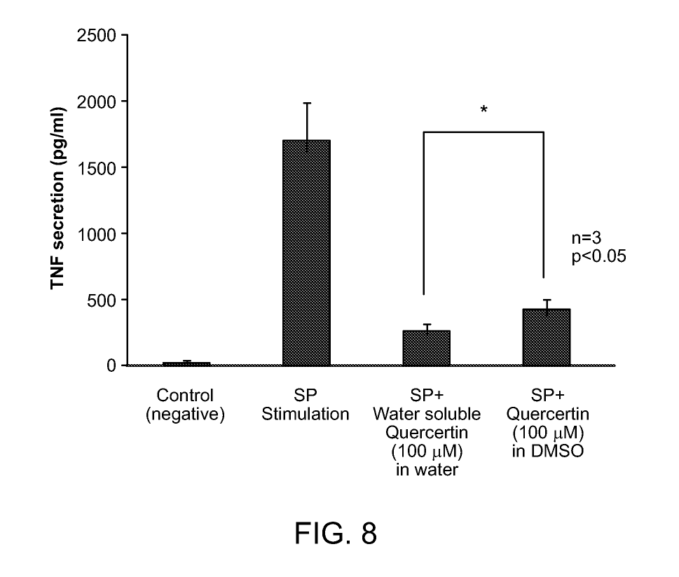

As can be seen in FIG. 8, increasing the water solubility of quercetin, increases the effectiveness of the flavanoid in suppressing TNF-alpha secretion. TNF-alpha is an important cytokine involved in inflammatory pathways in the body. The higher the TNF-alpha secretion the greater the inflammation. In the experiment described in FIG. 8, an equivalent amount (300μ) of anhydrous quercetin in Dimethyl sulfoxide and water soluble quercetin in water were administered. At the same concentration, water soluble quercetin shows better inhibitory effects compared with quercetin dissolved in DMSO. Substance P (SP) is a neuropeptide that is commonly used to stimulate mast cells serves as a positive control.

### Example 9

Table 1 Summarizes the Unique Benefits of the Components of CardioNiacin®.

TABLE 1
Unique benefits of CardioNiacin ®*

| Component | Actions | Side Effects |
|---|---|---|
| Niacin, extended release | ↓ cholesterol, ↓LDL, ↑HDL | Flush |
| | ↓ adipocytokines | |
| Luteolin | ↓ Inflammation, ↓ flush | N/A |
| | ↓ mast cell activation | |
| | ↓ Adipocyte-dependent | |
| | macophage activation, | |
| | Improves endothelial insulin | |
| | sensitivity | |
| Luteolin-glycoside | ↓ Cholesterol, ↓LDL | N/A |
| Quercetin | ↓ Inflammation, ↓ flush | N/A |
| | ↓ mast cell activation, ↓ CRP, | |
| | ↓ IL-6, ↓ PGD₂ | |
| | Mimics GLP-1 actions | |
| Olive kernel oil | ↓ Cholesterol, ↓ LDL | N/A |
| | ↑ Absorption of flavonoids | |

1. A pharmaceutical composition for protecting humans against superficial vasodilator flush syndrome (“SVFS”) comprising a flavonoid compound of Formula I: 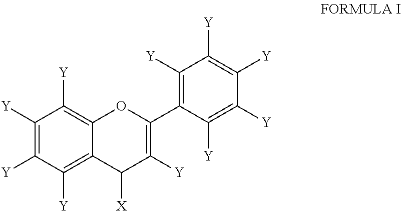
wherein X is carbonyl, O-Z, Y, or H;
each Y is independently H, O-Z, or a halogen;
Z is H, alkali metal, or alkali earth metal;
or a glycoside thereof.

2. The composition of claim 1, wherein said flavonoid compound is selected from the group consisting of luteolin, quercetin, myricetin, genistein, curcumin, epigallocatechin or a glycoside derivative of said flavonoids.

3. The composition of claim 1, wherein the flavonoid is formulated together with a phospholipid or cyclodextran to increase oral absorption.

4. The composition of claim 1, wherein said composition contains a composition that lowers total serum cholesterol or LDL cholesterol.

5. The composition of claim 4, wherein the cholesterol-lowering composition is a statin.

6. The composition of claim 5, wherein said statin is selected from the group consisting of simvastatin, I-vastatin, atorvastatin, rosuvastatin, fluvastatin, and provastatin.

7. The composition of claim 1, wherein said composition comprises of a compound that increases serum HDL cholesterol.

8. The composition of claim 1, wherein the composition contains a prostaglandin inhibitor.

9. The composition of claim 8, wherein the prostaglandin inhibitor is selected from the group consisting of non-steroidal anti-inflammatory drugs, COX-2 inhibitors, PGD₂ antagonist, and corticosteroids.

10. The composition of claim 9, wherein the PGD2 antagonist is laropiprant.

11. The composition of claim 1, wherein said flavonoid composition is supplemented with one or more additional anti-SVFS compounds.

12. The composition of claim 11, wherein said supplemental anti-SVFS compound is a serotonin inhibitor.

13. The composition of claim 12, wherein said inhibitor is a serotonin receptor antagonist.

14. The composition of claim 13, wherein said antagonist is prochlorperazine or ketanserin.

15. The composition of claim 13, wherein said serotonin inhibitor is a mixed histamine-1 and serotonin receptor antagonist selected from the group consisting of cyproheptadine or azatadine.

16. A pharmaceutical composition for protecting humans against superficial vasodilator flush syndrome (“SVFS”) comprising a chalconoid compound of Formula II: 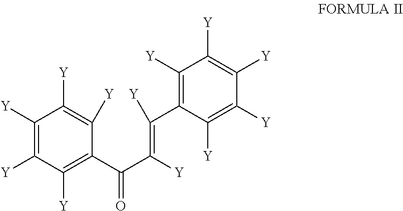
wherein each Y is independently H, O-Z, or a halogen;
Z is H, alkali metal, or alkali earth metal;
or a glycoside thereof.

17. The composition of claim 16, wherein said flavonoid compound is selected from the group consisting of luteolin, quercetin, myricetin, genistein, curcumin, epigallocatechin or a glycoside derivative of said flavonoids.

18. The composition of claim 16, wherein the flavonoid is formulated together with a phospholipid or cyclodextran to increase oral absorption.

19. The composition of claim 16, wherein said composition contains a composition that lowers total serum cholesterol or LDL cholesterol.

20. The composition of claim 19, wherein the cholesterol-lowering composition is a statin.

21. The composition of claim 20, wherein said statin is selected from the group consisting of simvastatin, I-vastatin, atorvastatin, rosuvastatin, fluvastatin, and provastatin.

22. The composition of claim 16, wherein said composition comprises of a compound that increases serum HDL cholesterol.

23. The composition of claim 16, wherein the composition contains a prostaglandin inhibitor.

24. The composition of claim 23, wherein the prostaglandin inhibitor is selected from the group consisting of non-steroidal anti-inflammatory drugs, COX-2 inhibitors, PGD₂ antagonist, and corticosteroids.

25. The composition of claim 24, wherein the PGD2 antagonist is Laropiprant.

26. The composition of claim 16, wherein said flavonoid composition is supplemented with one or more additional anti-SVFS compounds.

27. The composition of claim 26, wherein said supplemental anti-SVFS compound is a serotonin inhibitor.

28. The composition of claim 27, wherein said inhibitor is a serotonin receptor antagonist.

29. The composition of claim 28, wherein said antagonist is prochlorperazine or ketanserin.

30. The composition of claim 29, wherein said serotonin inhibitor is a mixed histamine-1 and serotonin receptor antagonist selected from the group consisting of cyproheptadine or azatadine.

31. A method for protecting an individual from the SVFS effects induced by niacin intake comprising administration to said individual effective doses for effective periods of time a composition of any one or more of claims 1 or 16.

32. A method for protecting an individual from SVFS-associated symptoms of superficial vasodilation, feeling of warmth, itching (pruritus) and hives comprising administration to said individual effective doses for effective periods of time a composition of any one or more of claims 1 or 16.

33. A method for protecting an individual from the SVFS effects associated with carcinoid-associated flush, mesenteric traction-induced flush, serotonin-induced flush, post-menopausal-induced flush, alcohol-induced flush and monosodium glutamate-induced flush, mastocytosis-induced, atopic dermatitis-induced, food-allergy or food intolerance-induced, and mast cell activation syndrome-induced SVFS comprising administration to said individual effective doses for effective periods of time a composition of any one or more of claims 1 or 16.

34. A method for treating a cardiovascular condition in a patient with niacin, but without eliciting the SVFS effect of said niacin, comprising the administration to said patient of clinically effective amounts of a composition of any one or more of claims 1 or 16.
